(19) United States
(12) Patent Application Publication
(10) Pub. No.: US 2025/0195755 A1
(43) Pub. Date: Jun. 19, 2025

(54) DEVICE FOR DELIVERING INSULIN INCLUDING BASEPLATE WITH INTEGRATED MEMS MICROPUMP AND FLUID CHANNELS

(71) Applicant: AITA BIO INC., New York, NY (US)

(72) Inventors: Dilan Casanovas Mack, New York, NY (US); Russell Cole, River Vale, NJ (US)

(21) Appl. No.: 18/837,858

(22) PCT Filed: Feb. 16, 2023
(86) PCT No.: PCT/US23/13181

§ 371 Date: Aug. 12, 2024

Related U.S. Application Data
(60) Provisional application No. 63/312,540, filed on Feb. 22, 2022.

(51) Int. Cl. A61M 5/142

(52) U.S. Cl. CPC A61M 5/14248; A61M 2205/0244; A61M 2205/0294

(57) ABSTRACT

A device for delivering medicament to a user is disclosed. The device is configured to be mounted to the user. The device comprises a baseplate including: an inlet opening and outlet opening to align with an inlet opening and outlet opening of a micropump, respectively; a first port for fluid in fluid communication with a catheter; and a first fluid channel to enable fluid communication between the first port and the outlet opening of the micropump; and a housing configured to engage the baseplate to form an interior therein, the interior including a first internal region that is sealed from fluid ingress and a second internal region that is not sealed from fluid ingress, wherein the inlet opening of the baseplate, the outlet opening of the baseplate and the first port are within the first internal region.

### CROSS REFERENCE TO RELATED APPLICATION

This application claims priority to U.S. provisional application 63/312,540, filed Feb. 22, 2022, entitled “Device For Delivering Insulin with Base Plate and Integrated MEMS Micropump” which is incorporated by reference herein.

### FIELD OF THE INVENTION

The present invention relates to a device for delivering insulin including a baseplate with integrated MEMS micropump and fluid channels.

### BACKGROUND OF THE INVENTION

Insulin pumps help people with diabetes to conveniently manage their blood sugar. These devices deliver insulin at specific times. Insulin patch pumps or pods are one type of insulin pump. The patch pumps are wearable devices that adhere to the skin of a user using an adhesive patch. The patch pumps are controlled wirelessly with a handheld controller. The patch pumps deliver insulin from a chamber and internal cannula based on separately acquired CGM sensor readings. The fluidic components within the patch pumps are typically significant in number to achieve proper fluid delivery. Component integration for such pumps are complex and current solutions are thus large and expensive. This complication risks device malfunction.

It would be advantageous to provide improvements to insulin pumps described above.

### SUMMARY OF THE INVENTION

A device for delivering insulin with base plate and integrated MEMS micropump is disclosed.

In accordance with an embodiment of the present disclosure, a device for delivering medicament to a user, the device configured to be mounted to the user, the device comprising: a baseplate including: an inlet opening and outlet opening to align with an inlet opening and outlet opening of a micropump, respectively; a first port for fluid in fluid communication with a catheter; and a first fluid channel to enable fluid communication between the first port and the outlet opening of the micropump; and a housing configured to engage the baseplate to form an interior therein, the interior including a first internal region that is sealed from fluid ingress and a second internal region that is not sealed from fluid ingress, wherein the inlet opening of the baseplate, the outlet opening of the baseplate and the first port are within the first internal region.

In accordance with another embodiment of the disclosure, a device for delivering medicament to a user, the device configured to be mounted to the user, the device comprising: (a) a baseplate and a housing configured to engage the baseplate to form an interior therein, the interior including a first internal region that is not sealed from fluid ingress; (b) a second internal region that is sealed from fluid ingress, wherein the baseplate includes a first opening that is within the interior, a second opening within the interior and a first fluid channel outside the interior communicating with the first and second openings, wherein the first opening, the second opening and first channel are within the second internal region sealed from fluid ingress, and wherein the medicament passes through the first channel from the first opening in the second internal region to the second opening in the second internal region.

In accordance with another embodiment of the disclosure, A device for delivering medicament to a user, the device configured to be mounted to the user, the device comprising: a micropump with an inlet opening and outlet opening; a baseplate including an inlet opening and outlet opening to align with the inlet opening and the outlet opening of a micropump, respectively; a reservoir in fluid communication with the inlet opening of the micropump; a catheter for delivering the medicament to the user; a fluid channel to enable fluid communication between the catheter and the outlet opening of the micropump; a first internal region that is sealed from fluid ingress; and a second internal region that is not sealed from fluid ingress, wherein the inlet opening of the baseplate, the outlet opening of the baseplate and the fluid channel are within the first internal region sealed from fluid ingress.

### BRIEF DESCRIPTION OF DRAWINGS

FIG. 1 depicts a perspective view of an example device for delivering insulin to the user.

FIG. 2 depicts a perspective view of a base plate of the device shown in FIG. 1.

FIG. 3 depicts a top view of a base plate of the device shown in FIG. 1 including an adhesive deposit.

FIG. 4 depicts a perspective exploded view of a base plate of the device shown in FIG. 1 along with micropump.

FIG. 5 depicts a perspective exploded view of a base plate of the device shown in FIG. 1 along with the micropump (bottom side).

FIG. 6 depicts a perspective view of a base plate of the device shown in FIG. 1 along with the integrated micropump installed.

FIG. 7 depicts a perspective bottom view of a base plate of the device shown in FIG. 1.

FIG. 8 depicts an exploded view of another example device for delivering insulin with a micropump integrated into the baseplate.

FIG. 9 depicts the device of FIG. 8 exposing internal components including the micropump integrated into the baseplate.

FIG. 10 depicts an exploded view of housing and baseplate of the device in FIG. 8.

FIG. 11 depicts a sectional top view of the device in FIG. 8 illustrating fluid channels in a heretically sealed region.

FIG. 12 depicts a sealing component and baseplate of the device in FIG. 8.

FIG. 13 depicts a block diagram of example components of a device for delivering insulin.

### DETAILED DESCRIPTION OF THE INVENTION

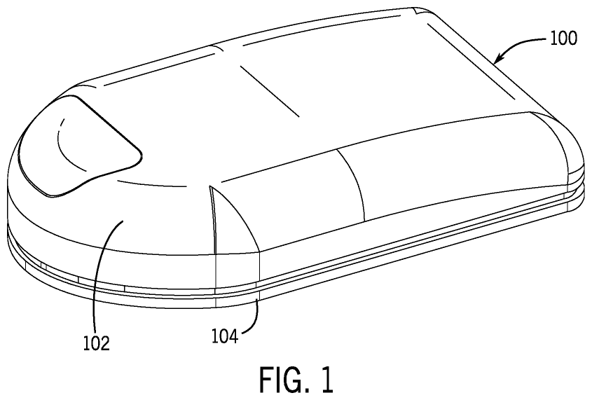

FIG. 1 depicts a perspective view of an example device 100 (or pod) for delivering insulin to the user. Device 100 (or pod) is configured as a wearable apparatus or system that is part of an infusion system for diabetes management in which continuous glucose monitoring (CGM), insulin delivery and control functionality are provided to ensure insulin is delivered at very precise rates. Device 100 however may be configured as a system of managing and infusing other medications/fluids to a user. Medication (also referred to as medicament) may include small molecule pharmaceutical solutions, large molecule or protein drug solutions, saline solutions, blood or other fluids known to those skilled in the art. Insulin is an example fluid and described below with respect to device 100. However, device 100 may be used in other environments known to those skilled in the art.

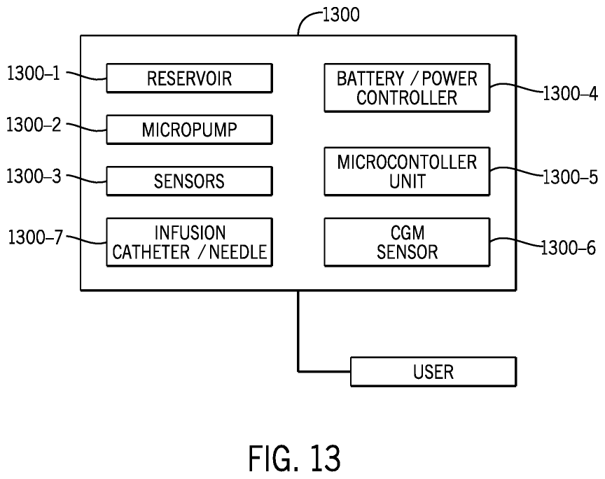

Device 100 includes incorporates several components or modules within housing 102 and base plate 104 such as micropump 106 as well as a reservoir for storing the insulin, control circuitry (integrated circuit—IC) such as a microcontroller unit (MCU), battery for powering the IC, an insulin catheter or needle and a continuous glucose monitoring (CGM) sensor (to name a few). FIG. 13 depicts an example device illustrating some of these components.

Micropump 106 (or micropump 802 below) may be a MEMS (micro-electro-mechanical systems) device, as known to those skilled in the art, that can be used for pumping fluid, valves used for regulating flow, actuators used for moving or controlling the micropump and valves and/or sensors used for sensing pressure and/or flow. The MEMS device incorporates one or more piezoelectric elements or devices (also known herein as piezoelectric transducers), as known to those skilled in the art. Example piezoelectric devices include piezoelectric actuators and various types of MEMS sensors. The piezoelectric devices function as the active element(s) of a pump for pumping fluid and valves for preventing fluid flow and/or a sensor for sensing pressure or flow. (However, various types of MEMS sensors can be used as the sensing elements of the architecture.) Further, the micropump 106 may be any other MEMS device. Alternatively, micropump 106 (or micropump 802 below) may be a non-MEMS structure or technology to achieve desired results as known to those skilled in the art.

Specifically, in this example, micropump 106 is a cavity substrate that includes a cavity defined by top and bottom wafers (e.g., silicon on insulator and silicon wafers or layers as known to skilled in the art). The top wafer functions as a membrane for the three chambers in this example (or could be any number of chambers). The bottom wafer includes inlet and outlet ports 108, 110 that communicate with valve chambers of the cavity via channels that extend through the bottom wafer. Micropump 106 includes pump section 112 and two valve sections 114, 116 that function together to pump fluid through the three chambers of the cavity.

Pump section 112 include piezoelectric actuator (transducers) 118 and valve sections 114,116 include piezoelectric actuators (transducers) 120,122 respectively that are layered as shown on the top wafer. A metallization and conductive epoxy layer may be used to bind piezoelectric actuators 108, 120 and 122 to the top wafer as known to those skilled in the art. Piezoelectric actuator 118 functions to pump or deform/bend the top wafer (silicon layer) to draw into or displace liquid contents into the pump cavity chamber from either port 108, 110 as desired. Micropump 106 also includes valve seats (not shown) that are configured to extend into valve cavity chambers and define the introduction of channels and inlet and outlet ports 108,110.

Valve sections 114, 116 are configured as piezoelectric microvalves that function as active valves as described in more detail below. Piezoelectric actuators 120,122 are configured to compress against the top wafer (membrane) to reach and seal the valve seats to thereby discontinue flow through inlet and outlets 108,110, respectively as needed for proper pump performance, as known to those skilled in the art. (Note that a micropump may include any number of pumps and/or valves as described herein).

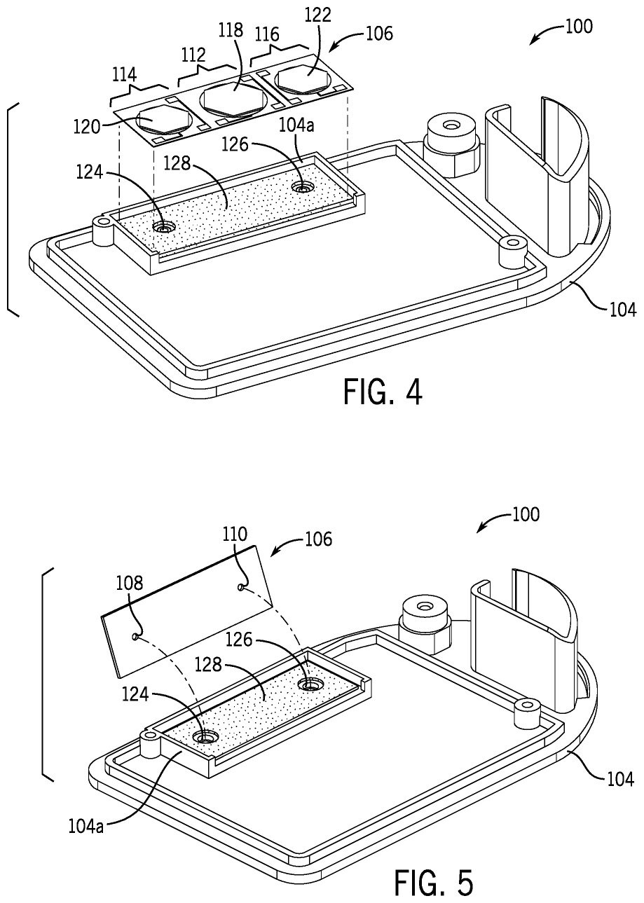

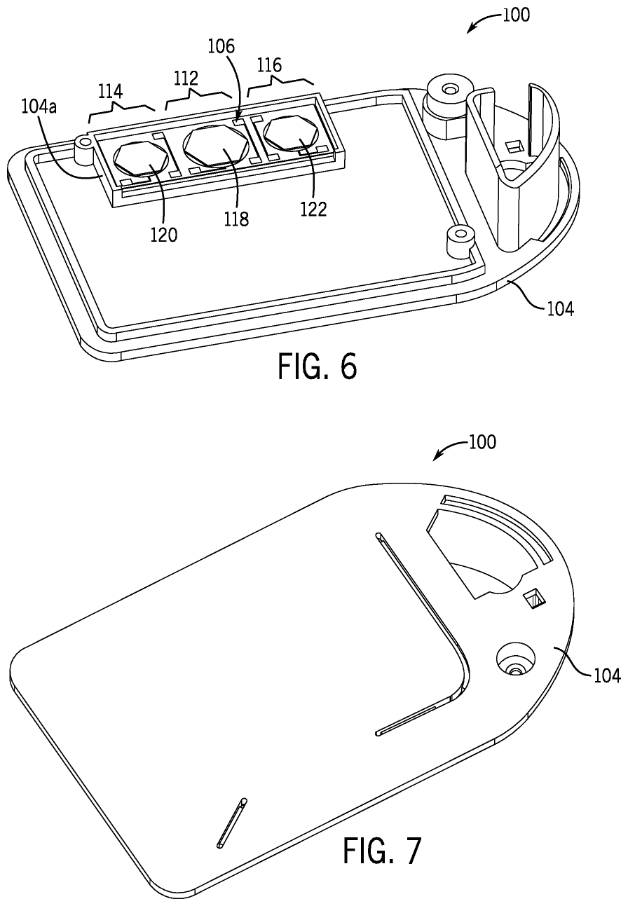

In the example shown in FIGS. 1-7, base plate 104 acts as the chassis of device 100 and it is configured to integrate micropump 106 onto such chassis itself. The fluidic connections are made between micropump 106 and platform 104a of baseplate 104. (Platform 104a is described in more detail below.) Baseplate 104 has the fluidic channels molded directly therein as shown so that when the bonding between micropump 106 and baseplate 104 is complete, there is no other step or structure necessary for the fluidic seal to be leak free. In this way, device 100 as disclosed herein combines MEMS packaging with the fluidic connections necessary to close the fluid path between a reservoir of the device 100 and the needle or cannula that delivers the medication (drug) to the patient. As a result, biocompatible fluidic connections are achieved and the baseplate of device 106 (medical device) is used as part of the bottom packaging of a micropump such as micropump 106.

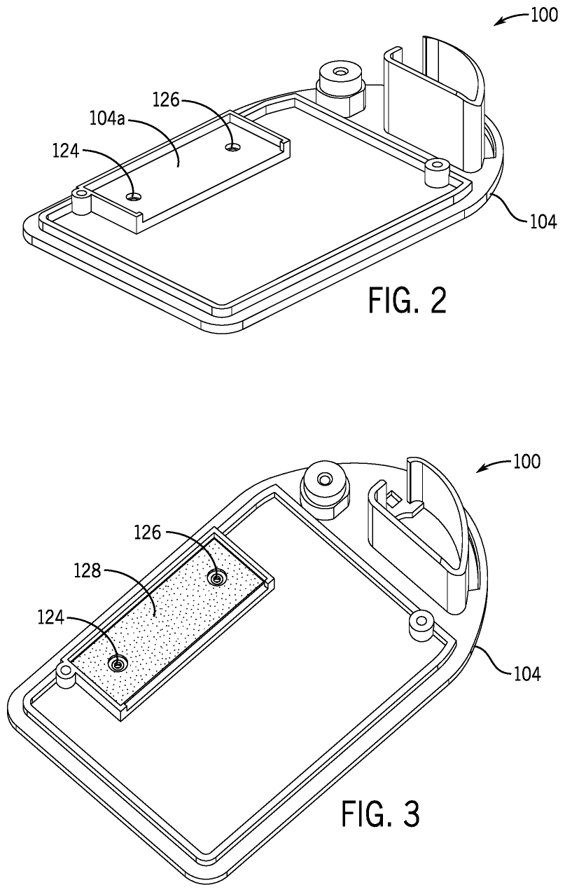

In more detail, baseplate 104 includes platform 104a described above. Platform 104a is configured in shape and dimension to receive and fit micropump 106 and to align inlet and outlet ports 108,110 on bottom of micropump 106 with corresponding outlet and inlet holes or openings/channels 124,126 (best seen in FIGS. 2-4) on baseplate 104. Outlet and an inlet holes 124, 126 are configured in the same position as inlet and the outlet ports 108, 110 (and channels) of micropump 106. That is, when micropump 106 is placed (mounted) onto platform 104a, outlet and inlet holes/channels 124, 126 align so that when the bonding occurs corresponding channels feed into each other.

As described above, the attachment of micropump 106 onto baseplate 104 serves two purposes. First, baseplate 104 acts as a base of the packaging and a protection layer. Second, a full integration of micropump 106 into the fluidic system of the overall medical device is achieved. As shown in in FIG. 3, biocompatible adhesive 128 is selectively deposited (as a layer) around the platform of baseplate 104 that is intended for micropump 106. The method of deposition can vary as known to those skilled in the art, but screen printing is an example of an efficient way to control the deposition, thickness and desired design. It is important that the adhesive is not deposited near outlet and inlet openings/channels 124,126 to avoid the material entering and blocking the fluid path. Once adhesive 128 has been deposited, micropump 106 is placed on top of platform 104a of baseplate 104, adhesive and micropump are cured to promote the adhesion and increase the strength of the bond between micropump 106 and baseplate 104. The adhesive can also be a biocompatible adhesive tape or film that is patterned with a hole and aligned and placed onto the baseplate platform 104a prior to attaching the micropump (e.g., MEMS device or chip) to it. The adhesives can be separated in two (one for the inlet and one for the outlet). This facilitates leakage testing to identify any fluidic leaks between the two channels.

Finally, the fluidic channels on the bottom of baseplate 104 are sealed by heat staking a film to it that covers the channels. A tube is connected from the reservoir to opening/channel 126 of baseplate 104 that leads to inlet 108 of micropump 106. Once the drug fluid is pumped out of micropump 106 (e.g., MEMS device or chip), it travels through the channel that ends up in a tubing that is connected to the needle/cannula of device 100.

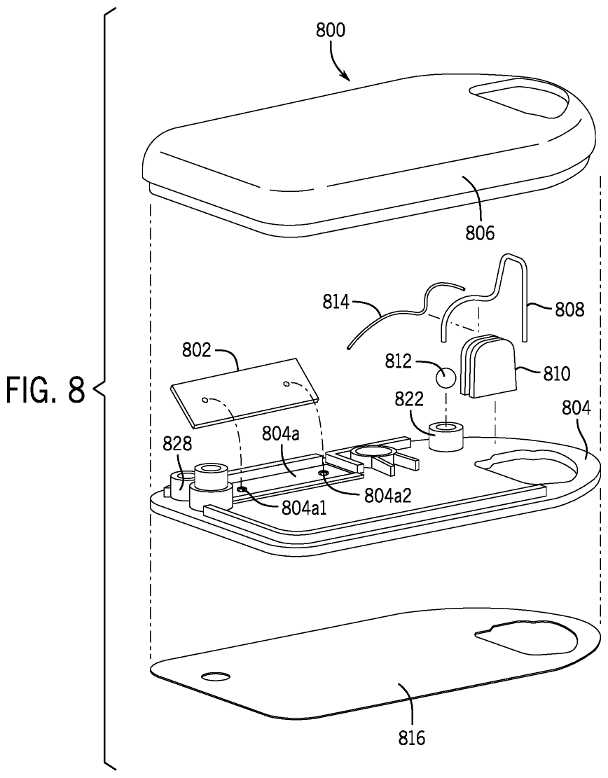

FIG. 8 depicts an exploded view of another example device 800 for delivering insulin with a micropump 802 integrated into baseplate 804. Similar to device 100, device 800 includes housing 806 that engages baseplate 804 to define an interior of device 800. Housing 806 functions as a top cover for baseplate 804. Device 800 includes catheter 808 (or tubing connected to catheter 808), catheter and sensor wire seal 810, fluid path septum 812, CGM connection wires 814 and fluid channel sealing sheet 816.

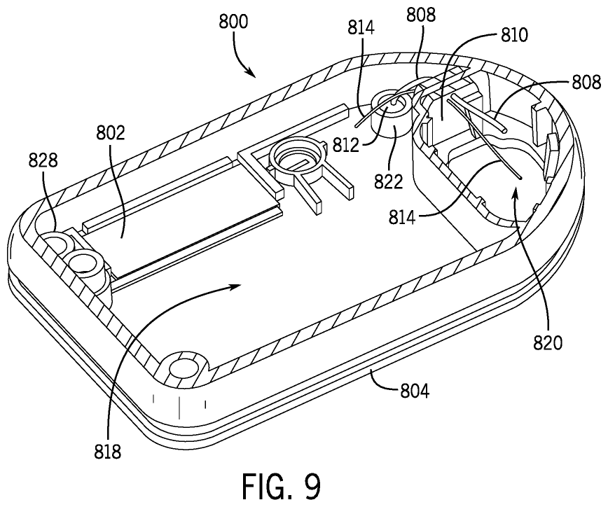

FIG. 9 depicts the device shown in FIG. 8, exposing internal components including micropump 802 (similar to micropump 106 above) integrated into baseplate 804 (as shown in example device 100 described above). Specifically, baseplate 804 includes platform 804a to receive or support micropump 802. Platform 804a has a similar shape and dimension as micropump 802 so that it fits nicely into it. The openings or holes of platform 804a are aligned with the openings or holes of the bottom of micropump 802 so that when bonding occurs, the channels feed into each other. The fluidic connections are thus made between micropump 802 and the baseplate 804. Baseplate 802 has the fluidic channels molded into it. No other steps are necessary for the fluidic seal (leak free). Attaching the micropump 802 onto baseplate 804 serves dual purposes. First, the baseplate acts as the base of the micropump 802 packaging and a protection layer. Second, integration of micropump 802 into the fluidic system of device 800 reduces additional components as a fluidic pathway. A biocompatible adhesive is selectively deposited around platform 804a. Alternatively, O-rings are selectively deposited to encircle the inlet and outlet openings in the MEMS pump in order to seal the fluid from contacting the adhesive.

Device 800 incorporates hermetically sealed region 818 and a non-hermetically sealed region 820. Housing 806 and baseplate 804 define an interior that includes non-hermetically sealed region 820. Hermetically sealed region 818 is encompassed in part within the interior and in part outside the interior. Stated differently, hermetically sealed region 818 includes a third region or sub-region that is outside the interior (opposing side of baseplate 804). The third region encompasses fluid channels 824,826 (described below) that are sealed by sealing sheet 816.

A fluid path is molded into baseplate 804 and catheter 808 is connected to port 822 that is within hermetically sealed region 818 of the device 800. Catheter 808 extends through fluid path septum 812 as shown. Catheter 808 passes through hermetic seal region 818 via a slit in a seal 810 (elastomeric sealing component) that is trapped between top cover housing 806 and baseplate 804. The seal is compressed, but top cover housing 806 and baseplate 804 actually create a watertight seal around catheter 808. One or more wires 814 that connects a CGM sensor which is located in region 820 (outside of the hermetically sealed region 818 of the device 800) to the electronics inside the hermetically sealed region 818 may be sealed by the same seal 810 (elastomeric component). The sealing of catheter 808 and/or sensor may utilize an adhesive in addition to the sealing component or adhesive alone without the sealing component. As part of hermetically sealed region 818, catheter 808 extends through septum 812 within port 822 as shown.

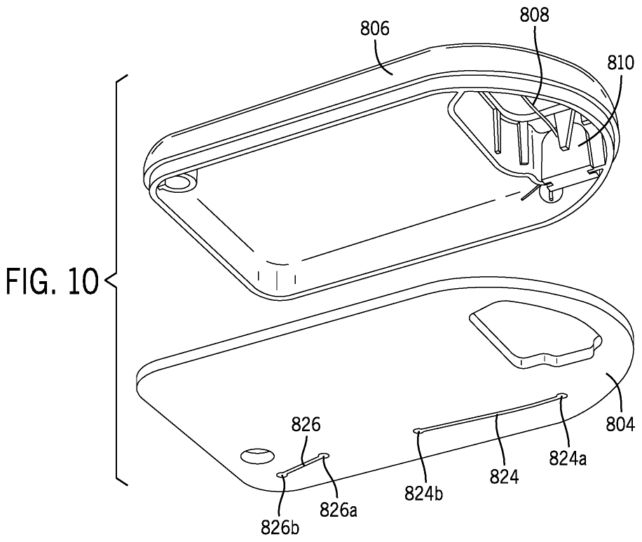

FIG. 10 depicts an exploded view of housing 806 and baseplate 804 of the device in FIG. 8. Device 800 is shown before hermetically sealed region 818 is created and catheter 808 is connected to baseplate 804. Seal 810 (elastomeric sealing component) is shown. Fluid channels 824, 826 are molded (recessed) into the bottom of baseplate 804. Fluid channel 824 has passageway 824a that extends through baseplate 804 and communicates with catheter 808 through and opening in port 822 (of baseplate 804) and passageway 824b that extends through baseplate 804 and communicates with micropump 802 through opening 804a1 in platform 804a (FIG. 8). Fluid channels 824,826 are outside the interior of device 800. Fluid channel 826 also has passageways 826a,826b. Passageway 826a extends through baseplate 804 and communicates with micropump 802 through opening 804a2 in platform 804a and passageway 826b extends through baseplate 804 and communicates to a reservoir (not shown in FIGS. 8-12) via an opening in port 828 and another septum. Channels 824 and 826 reside in the hermetically sealed region 818 of device 800. Fluid channel sealing sheet 816 is configured to cover and seal the entire bottom surface of baseplate 804 and channels 824,826.

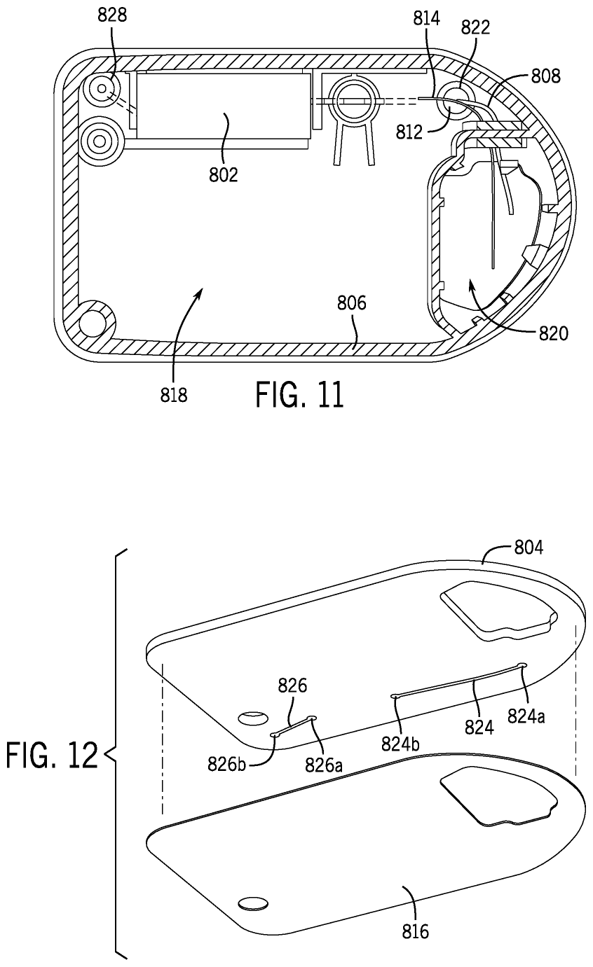

FIG. 11 depicts a sectional top view of the device in FIG. 8 illustrating fluid channels 824,826 in a heretically sealed region. FIG. 12 depicts a sealing component and baseplate of the device in FIG. 8.

In the example device 800 in FIGS. 8-12, hermetically sealed region 818 is larger region than example device 100 shown in FIGS. 1-7 to include more components including port 822, septum 812, and catheter 808 as shown. In other words, by enlarging the hermetically sealed region 818 to include port 822 and other components, volume in the non-hermetically sealed region 820 has been reduced. Thus, fluid build-up from user sweat, showers etc. in non-hermetically sealed region 820 is reduced.

FIG. 13 depicts a block diagram of example components of device 1300 for delivering insulin of an infusion system as described above. (Device 100 or device 800 is represented as device 1300 in FIG. 13.) Specifically, device 1300 incorporates several components or modules (not shown) in the fluidic pathway including reservoir 1300-1 for storing the insulin, micropump 1300-2 (as described hereinabove) for pumping the insulin or other medicament, sensors 1300-3 (e.g., pressure) for sensing various parameters in the system and user and tubing connecting infusion catheter or infusion needle 1300-7 to reservoir 1300-1. Device 1300 also includes microcontroller unit (MCU) 1300-4 and battery and power controller 1300-5.

Device 1300 further includes CGM sensor 1300-6. CGM or continuous glucose monitoring, as known to those skilled in the art, tracks user glucose levels and permits those levels to be used in algorithms that control flow rate. MCU 1300-4 controls the operation of micropump 1300-2. Infusion needle 1300-7 and CGM sensor 1300-6 are shown as separate components in FIG. 4 for illustration purposes. Infusion catheter or needle 1300-7 and CGM sensor 1300-6 may be integrated or may be separate (individually).

Reservoir 1300-1 is configured to receive and store insulin (or other medicament) for its delivery over a course of about three days, or as needed. However, reservoir size may be configured for storing any quantity of fluid as required.

MCU 1300-5 electronically communicates with sensors 1300-3 and micropump 1300-2 as well as the CGM sensor 1300-6, as the monitoring components. Among several functions, MCU 1300-5 operates to control the operation of micropump 1300-2 to deliver insulin through infusion catheter or infusion needle 1300-7 from reservoir 1300-1 at specific doses, i.e., flow rates over specified time intervals, based on CGM data converted to desired flow rate via control algorithms.

Battery and power controller 1300-4 controls the power to MCU 1300-5 and micropump 1300-2 to enable those components to function properly as known to those skilled in the art. CGM sensor 1300-2 is powered by battery and power controller 1300-4 through MCU 1300-5.

FIG. 13 depict device 1300 with only a few components. Those skilled in the art know that device 1300 include additional components.

It shall be understood that this disclosure teaches examples of the illustrative embodiments and that many variations of the invention can easily be devised by those skilled in the art after reading this disclosure and that the scope of the present invention is to be determined by the claims below.

What is claimed is:

1. A device for delivering medicament to a user, the device configured to be mounted to the user, the device comprising:
(a) a baseplate including:
an inlet opening and outlet opening to align with an inlet opening and outlet opening of a micropump, respectively;
a first port for fluid in fluid communication with a catheter; and
a first fluid channel to enable fluid communication between the first port and the outlet opening of the micropump; and
(b) a housing configured to engage the baseplate to form an interior therein, the interior including a first internal region that is sealed from fluid ingress and a second internal region that is not sealed from fluid ingress, wherein the inlet opening of the baseplate, the outlet opening of the baseplate and the first port are within the first internal region.

2. The device of claim 1 further comprising a seal between the first internal region and second internal region.

3. The device of claim 2 wherein the seal is configured to enable the catheter to pass from the first internal region to the second internal region to enable the catheter to deliver medicament to the user via the second internal region while maintaining the seal of the first internal region.

4. The device of claim 1 wherein the outlet opening of the micropump and the first port define ends of the first fluid channel.

5. The device of claim 4 further comprising sealing sheet for sealing the fluid channel.

6. The device of claim 1 wherein the first fluid channel passes from a first position in the interior to a second position outside the interior to a third position in the interior.

7. The device of claim 6 wherein the first position corresponds to first internal region and the third position corresponds to first internal region.

8. The device of claim 1 wherein the baseplate further including a second port and a second fluid channel enabling fluid communication between inlet opening of the micropump of the second port.

9. The device of claim 1 wherein the medicament is insulin.

10. A device for delivering medicament to a user, the device configured to be mounted to the user, the device comprising:
(a) a baseplate and a housing configured to engage the baseplate to form an interior therein, the interior including a first internal region that is not sealed from fluid ingress;
(b) a second internal region that is sealed from fluid ingress,
wherein the baseplate includes a first opening that is within the interior, a second opening within the interior and a first fluid channel outside the interior communicating with the first and second openings,
wherein the first opening, the second opening and first channel are within the second internal region sealed from fluid ingress, and
wherein the medicament passes through the first channel from the first opening in the second internal region to the second opening in the second internal region.

11. The device of claim 10 wherein the first fluid channel is recessed into the baseplate outside the interior.

12. The device of claim 10 wherein the first fluid channel includes first and second passageways that extends through the baseplate and communicate with the first opening and second opening, respectively.

13. The device of claim 10 further comprising a seal sheet over the first channel thereby sealing the first channel.

14. The device of claim 10 wherein the baseplate further includes a first port in the first internal region that is in fluid communication with first channel via the first opening in the baseplate.

15. The device of claim 10 further including a seal for separating the first internal region from the second, whereby the seal is configured to enable the catheter to pass from the second internal region to the first internal region to deliver the medicament to the user while maintaining the seal of the second internal region.

16. The device of claim 14 wherein the base plate includes a third opening in the interior, a fourth opening in the interior and a second fluid channel outside the interior in fluid communication with the third and fourth openings, wherein the third opening, the fourth opening and the second channel section are within the second internal region sealed from fluid ingress.

17. The device of claim 16 wherein the baseplate includes a second port in the second internal region that is fluid communication with the second fluid channel through the fourth opening in the baseplate.

18. The device of claim 16 wherein the second opening in the baseplate and fourth opening in the baseplate are in communication with an inlet opening and an outlet opening of a micropump, respectively.

19. The device of claim 10 wherein the second internal region includes a third region outside the interior.

20. The device of claim 19 wherein the third region encompasses channel section.

21. The device of claim 10 wherein the medicament is insulin.

22. A device for delivering medicament to a user, the device configured to be mounted to the user, the device comprising:
a micropump with an inlet opening and outlet opening;
a baseplate including an inlet opening and outlet opening to align with the inlet opening and the outlet opening of a micropump, respectively;
a reservoir in fluid communication with the inlet opening of the micropump;
a catheter for delivering the medicament to the user;
a fluid channel to enable fluid communication between the catheter and the outlet opening of the micropump;
a first internal region that is sealed from fluid ingress; and
a second internal region that is not sealed from fluid ingress,
wherein the inlet opening of the baseplate, the outlet opening of the baseplate and the fluid channel are within the first internal region sealed from fluid ingress.

23. The device of claim 22 wherein the baseplate includes a platform for supporting the micropump.

24. The device of claim 22 wherein the fluid channel is recessed within the baseplate.

25. The device of claim 22 wherein the micropump is a MEMS device.
